(19) United States
(12) Patent Application Publication
(10) Pub. No.: US 2009/0112209 A1
(43) Pub. Date: Apr. 30, 2009

(54) IMPLANTATION SYSTEM FOR INTRAMEDULLARY NAIL AND RELATED METHODS FOR IMPLANTING INTRAMEDULLARY NAILS

(75) Inventors: Rebecca Parrott, Winona Lake, IN (US); Michael Kay, Peru, IN (US); Lynn Stanley, Columbia City, IN (US); Michael Wack, Warsaw, IN (US); Daniel Buehler, Warsaw, IN (US)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER, INC., Warsaw, IN (US)

(21) Appl. No.: 11/931,243

(22) Filed: Oct. 31, 2007

(51) Int. Cl. A61F 2/30; A61B 17/56; A61B 17/58

(52) U.S. Cl. 606/62; 606/96; 606/104

(57) ABSTRACT

An intramedullary implantation system includes a nail with a head having a first locking element. A targeting guide includes a tubular body and a bore with first and second open ends. A locking device includes a shaft with a distal end having a non-threaded second locking element such that the locking device has a first angular orientation relative to the tubular body in which the first and second locking elements are engaged to secure the nail to the targeting guide. The locking device has a second angular orientation relative to the tubular body in which the first locking element is disengaged from the second locking element.

### FIELD OF THE INVENTION

The invention relates generally to intramedullary implants and, more particularly, to systems and methods for implanting intramedullary nails.

### BACKGROUND OF THE INVENTION

Intramedullary nails or rods are used for procedures such as fracture fixation. Known approaches for implantation of intramedullary nails include the coupling of the nail to a targeting guide. Targeting guides are used, for example, to facilitate impaction surfaces to drive the nail into a desired target. Likewise, targeting guides may be used to provide alignment for drilling transverse bores through a bone to accommodate screws to lock the nail to the bone.

Coupling of the nail to the targeting guide may include the insertion of a locking device in the form of a threaded locking bolt. Such locking bolts may present various deficiencies, such as loosening thereof during the implantation procedure.

Accordingly, an implantation system and related methods are needed that address this and other deficiencies of conventional systems and methods for implanting intramedullary nails.

### SUMMARY OF THE INVENTION

The systems and methods associated with the various embodiments of this invention are used to aid in the implantation of intramedullary nails. In one embodiment, an intramedullary implantation system includes a nail, a targeting guide, and a locking device. The nail includes with a head having a first locking element. The targeting guide includes a tubular body with a first open end, a second open end, and a bore extending between the first and second open ends. The locking device includes a shaft with a distal end and a non-threaded second locking element on the distal end. The distal end is located proximate to the first open end of the bore of the targeting guide, when the shaft is positioned in the bore, so that the second locking element is engagable with the first locking element. The locking device has a first angular orientation relative to the tubular body in which the first and second locking elements are engaged to secure the head of the nail to the targeting guide, and a second angular orientation relative to the tubular body in which the first locking element is disengaged from the second locking element.

The use of the non-threaded locking elements eliminates the need for an external instrument or device, such as a screwdriver, to couple the nail with the targeting guide. The locking elements produce a positive lock with the nail that ensures that the targeting guide and the nail are securely locked together. When the locking elements are secured together, the locking device cannot be retracted.

In a specific embodiment, the first and second locking elements jointly define a twist lock. The second locking element may further include a male component of the twist lock. The second locking element may also include at least one tab configured to contact a confronting surface of the first locking element to bring the nail, the targeting guide, and the locking device into locking engagement with one another.

In specific embodiments, the tab includes a cam surface configured to contact the confronting surface. The cam surface may be further configured as a bidirectional cam surface adapted to allow selective rotation of the second locking element in a clockwise direction or a counterclockwise direction to bring the nail, the targeting guide, and the locking device into locking engagement with one another.

In another specific embodiment, a biasing member is positioned between the targeting guide and the locking device, and is configured to bias the nail toward the targeting guide. The biasing member may, for example, take the form of a cupped spring washer.

In another specific embodiment, the locking device includes a first ancillary locking element and the targeting guide includes a second ancillary locking element. The second ancillary locking element is configured to cooperate with the first ancillary locking element to bring the targeting guide and the locking device into locking engagement with one another. The first ancillary locking element may, for example, take the form of a protruding male member and the second ancillary locking element may take the form of a protruding female member.

The first ancillary locking element may be disposed on a handle portion of the locking device while the second ancillary locking element may be disposed on an extending portion of the targeting guide. In a specific embodiment, the first ancillary locking element is configured to engage the second ancillary locking element when the locking device is rotated relative to the targeting guide.

In another specific embodiment, the system may include a redundant locking mechanism configured to restrict axial and rotational movement of the targeting guide relative to the locking device. An impaction plate may be coupled to the locking device and be configured to receive and transfer a driving force onto the nail.

The locking device may include a handle portion pivotally coupled to the shaft thereof and having a pivot axis and a cam surface configured to apply a force against a confronting surface on the targeting guide. Rotation of the handle portion about the pivot axis engages the cam surface with the confronting surface to bring the targeting guide and the locking device into locking engagement with one another.

In another embodiment, an intramedullary nail includes a longitudinal axis and a shank disposed about the longitudinal axis. A head is coupled to the shank and is configured for inserting a locking element therein in a first angular orientation relative to the head. The head is also configured to allow rotation of the locking element to a second angular orientation to restrict the nail and the locking element from movement relative to one another along the longitudinal axis. The head may include a female component of a twist lock.

In yet another embodiment, a method of securing a targeting guide to an intramedullary nail includes coupling the targeting guide to a head of the nail. A locking device in inserted through a bore of the targeting guide and a non-threaded end of the locking device is rotated from a first angular orientation relative to the targeting guide to a second angular orientation relative to the targeting guide to secure the targeting guide to the nail.

A cammed portion of the locking device may be rotated to secure the targeting guide to the nail. This step may, for example include rotating a handle portion of the locking device about a pivot axis intersecting a longitudinal axis of the locking device. This step may also include rotating a distal end of the locking device about the longitudinal axis of the locking device.

### BRIEF DESCRIPTION OF THE DRAWINGS

The above-mentioned and other features and advantages of the embodiments of the invention, and the manner of attaining them, will become more apparent and the invention itself will be better understood by reference to the following description of embodiments of the invention taken in conjunction with the accompanying drawings, wherein:

FIG. 1 is a partial, disassembled perspective view of an implantation system for an intramedullary nail in accordance with one embodiment of the invention;

FIG. 2 is a partial, assembled perspective view of the implantation system of FIG. 1;

FIG. 3A is a partial perspective view of respective distal ends of the locking device and targeting guide of the system of FIG. 1;

FIG. 3B is a partial perspective view of the distal ends of FIG. 3A showing rotation of the locking device relative to the targeting guide;

FIG. 4 is a cross-sectional view taken generally along line 4-4 of FIG. 2;

FIG. 5 is a partial elevational view of a tab and cam surface of the locking device of FIGS. 3A-3B;

FIG. 5A is a view similar to FIG. 5 of a tab and cam surface in accordance with an alternative embodiment of the invention;

FIG. 5B is a view similar to FIG. 5 of a tab and cam surface in accordance with an alternative embodiment of the invention;

FIG. 5C is a cross-sectional view similar to FIG. 4, but with the locking device and the targeting guide omitted for clarity, of an intramedullary nail including a cam surface in accordance with an alternative embodiment of the invention;

FIG. 6 is a view similar to that of FIG. 2 with the addition of a redundant locking element;

FIG. 7 is a partial, disassembled perspective view of another embodiment of an implantation system for an intramedullary nail;

FIG. 8 is a partial elevational view of a tab of a locking device of the system of FIG. 7;

FIG. 9 is a partial assembled perspective view of the system of FIG. 7;

FIG. 10 is a partial assembled perspective view of the system of FIGS. 7 and 9 showing the addition of a redundant locking element;

FIG. 11A a disassembled perspective view of an implantation system for an intramedullary nail in accordance with an alternative embodiment of the invention and in which the cam surface is present on the handle portion of the locking device;

FIG. 11B is a partial perspective view of the implantation system of FIG. 11A with the handle portion of the locking device in a position that secures the intramedullary nail with the locking device and targeting guide;

FIG. 12 a partially broken away, partial perspective view of an implantation system for an intramedullary nail in accordance with an alternative embodiment of the invention and in which the cam surface is present on the inner diameter of the bore of the targeting guide; and

FIG. 13 a partially broken away, partial perspective view of an implantation system for an intramedullary nail in accordance with an alternative embodiment of the invention and in which the cam surface is present on the locking device near the handle portion.

### DETAILED DESCRIPTION

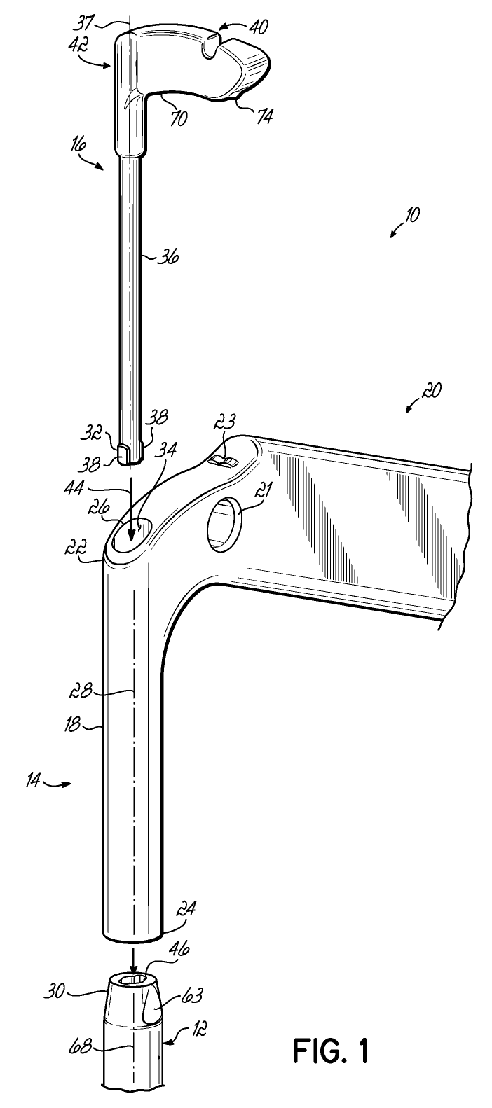

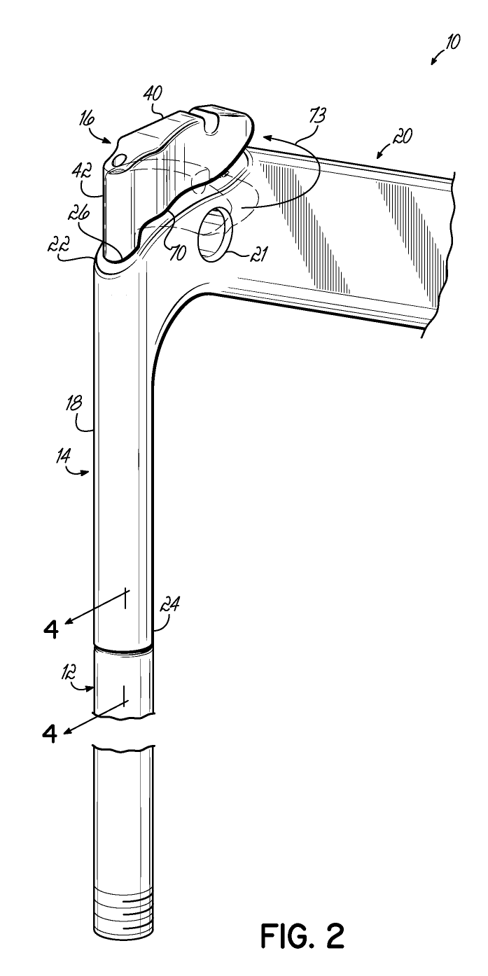

With reference to FIGS. 1-2, an implantation system 10 includes an implant in the representative form of an intramedullary nail 1 2, a targeting guide 14 configured for coupling to the nail 12, and a locking device 16 to facilitate coupling of the targeting guide 14 and nail 12. The targeting guide 14, which generally has an inverted L-shape, may further include, for example, and without limitation, drill guide bores and the like (not shown) to facilitate, among others, fastening or coupling of the nail 12 to other components such as screws (not shown). Characteristics and features of an exemplary targeting guide for use with the embodiments herein described are described, in detail, in commonly-assigned U.S. Pat. No. 5,478,341, the disclosure of which is hereby incorporated by reference herein in its entirety.

The targeting guide 14 includes a tubular body or barrel 18 and an extending portion 20 extending laterally from a proximal end 22 of the barrel 18. The extending portion 20 may include, as explained in further detail below, an opening 21 and an ancillary locking element in the form of a cusped locking ridge 23, both of which facilitate locking engagement of the targeting guide 14 and nail 12. A distal end 24 of the barrel 18 is engageable with the nail 12, as explained in further detail below. A bore 26 extends between the ends 22, 24 of the barrel 18 and defines a longitudinal axis 28. The bore 26 is sized to facilitate coupling of targeting guide 14 with the nail 12. More particularly, the size of the bore 26 is such that it may at least partially accept a head 30 of the nail 12 therein. To this end, the head 30 of the nail 12 may be tapered inwardly such as to cooperate with bore 26 to facilitate full receipt of the head 30 into a portion of the bore 26, as best depicted in the assembled cross-sectional view of FIG. 4.

Bore 26 is further sized to receive at least a portion of the locking device 16 therethrough. More particularly, bore 26 is sized to receive at least a non-threaded distal end 32 of the locking device 16. Moreover, bore 26 may be shaped such that the distal end 32 can be inserted into and travel through bore 26 in one or more discrete orientations. For example, and without limitation, bore 26 may be shaped such that the distal end 32 of the locking device 16 can be inserted in only one orientation, namely, the illustrative orientation depicted in the embodiment of FIG. 1. Accordingly, the bore 26 may include recesses 34 that guide the distal end 32 of the locking device 16 from the proximal end 22 to the distal end 24 of the barrel 18.

The locking device 16 facilitates coupling and, more particularly, locking engagement at least in an axial direction i.e., along axis 28 of the barrel 18, of the targeting guide 14 and nail 12. To this end, locking device 16 includes a shaft 36 defining an axis 37, one or more tabs 38 at the distal end 32 thereof, and a handle portion 40 at a proximal end 42 of the shaft 36. In the exemplary embodiment of FIG. 1, locking device 16 is depicted including two such tabs 38, although persons of ordinary skill will readily appreciate that tabs 38 in any other suitable number in excess of or less than two may be alternatively present. The tabs 38 are raised from the surrounding surface of the shaft 36.

The locking device 16 may be composed of a stable, radiolucent material. Alternatively, the locking device 16 may be made of a single use radiolucent device or an over mold with tabs made from a metal.

At least a portion of the shaft 36 is inserted through bore 26 of targeting guide 14 in a direction as shown by arrow 44. When such insertion is carried out after coupling of the targeting guide 14 and nail 12, as explained above, the distal end 32 of locking device 16 is further received within an aperture 46 in the head 30 of nail 1 2. To this end, the aperture 46 defines a shape that is configured to receive a corresponding cross-sectional shape of the distal end 32 and which is at least partially defined by the tabs 38. Moreover, the aperture 46 may be shaped such that it closely resembles the cross-sectional shape of the distal end 32. Accordingly, aperture 46 may be shaped such that the distal end 32 may be received in only one discrete orientation, as depicted in the exemplary embodiment of FIG. 1 and better appreciated in the cross-sectional view of FIG. 4.

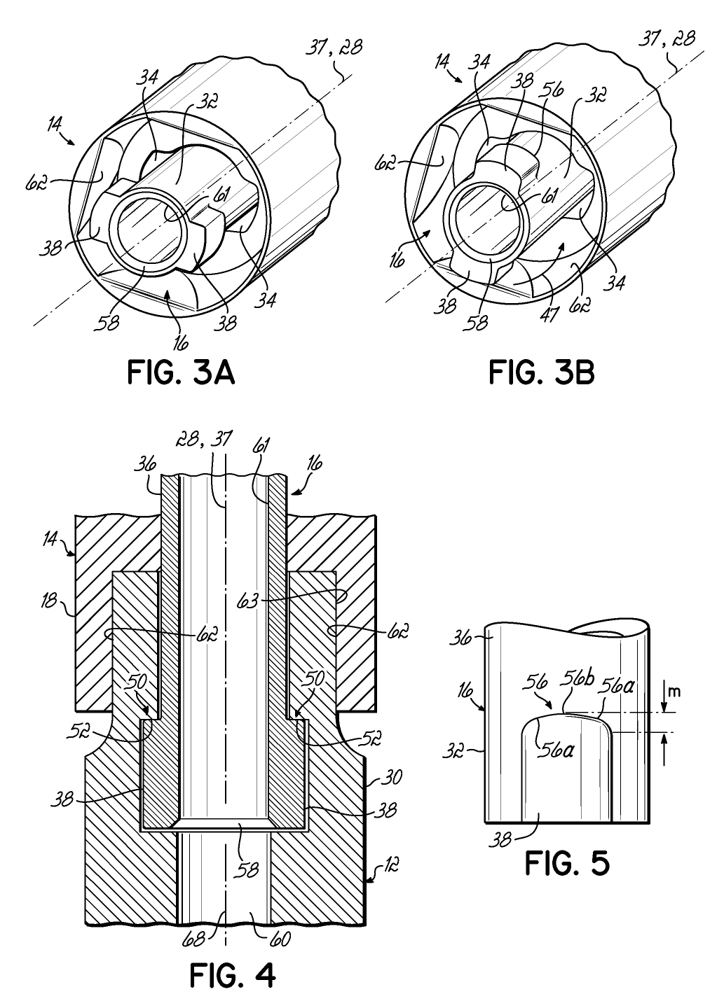

With reference to FIGS. 3A, 3B, 4 and 5, once received in the aperture 46, the locking device 16 can be lockingly coupled or engaged with the nail 12. More particularly, the locking device 16 is generally rotatable about axis 37 between first and second angular orientations relative to the head 30 and the barrel 18 and respectively corresponding to the depicted orientations in FIGS. 1 and 2. Rotation of the locking device 16 about axis 37, accordingly, is such that the tabs 38 at the distal end 32 can engage corresponding locking portions 50 of the head 30 of nail 12. When rotated about axis 37, as in the direction indicated by arrow 47, each of the tabs 38 abuts against a corresponding confronting surface 52 of each locking portion 50, thereby restricting axial motion (i.e., in a direction along axis 28 of the targeting guide 14 relative to the nail 12). Accordingly, coupling of the locking device 16 and nail 12 jointly define a twist lock, wherein the configuration of distal end 32 and head 30 respectively define a male and a female component of the twist lock.

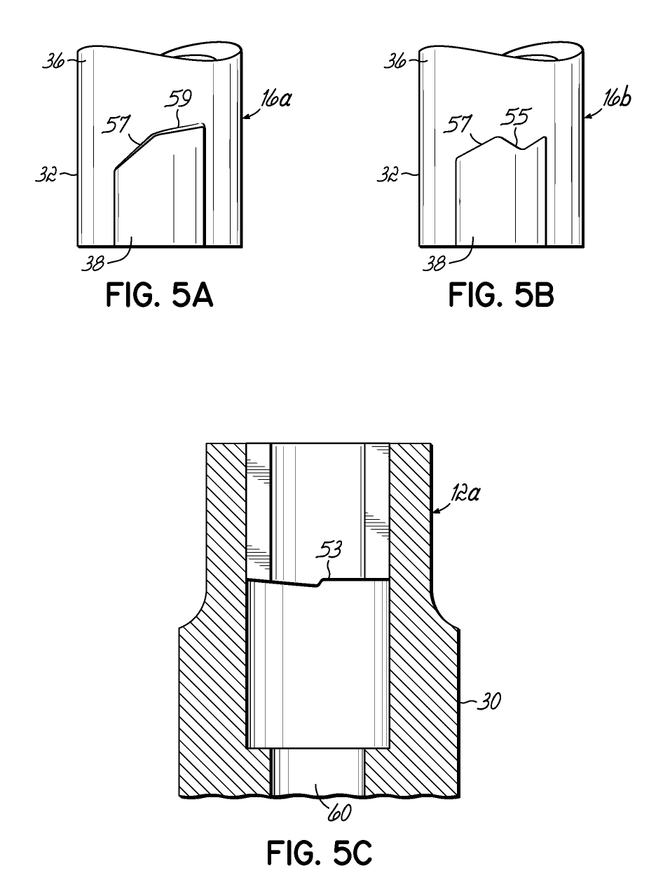

With particular reference to FIGS. 4 and 5, each tab 38 includes a cam surface 56 that abuts against the confronting surfaces 52 of locking portion 50. When engaged, the cam surface 56 further lockingly engages the locking device 16 to the nail 12. In one aspect of this embodiment, frictional engagement of the cam surface 56 with confronting surfaces 52 secure the angular orientation of the tabs 38 relative to locking portion 50. This therefore secures the angular orientation of the locking device 16 relative to the nail 12. More particularly, rotation of the locking device 16 e.g., in the direction of arrow 47, further advances the nail 12 and locking device 16 toward one another, at least by an amount equivalent to a rise, m, of the cam surface 56. The rise, m, displaces the nail 12 and locking device 16 toward the open end 22 of the barrel 18 of targeting guide 14, which provides a relatively secure assembly that can be used to implant the nail 12 in a patient.

Cam surface 56 is further configured as a bidirectional cam surface, including inwardly rising portions 56a on each side of an apex or peak 56b. Accordingly, the locking device 16 can be engaged with or disengaged from nail 12 by rotating locking device 16 in either a clockwise direction or a counterclockwise direction (e.g., in the direction of arrow 47 of FIG. 3B or a direction opposite thereto).

In alternative embodiments, the cam surface 56 may have different constructions understood by a person having ordinary skill in the art. For example, the cam surface 56 may be unidirectional, rather than bidirectional, and include only one of the rising portions 56a. As a specific example and as shown in FIG. 5A, a unidirectional cam surface of a locking device 16a is contoured to include two different inclines 57, 59 such that the torsion required to raise the shaft 36 up a first incline 57 differs from the torsion required to raise the shaft up a second inclined 59 that adjoins the first incline 57. As another specific example and as shown in FIG. 5B, a unidirectional cam surface of a locking device 16b includes the incline 57 and a notch 55, instead of incline 59 (FIG. 5A), that is adjacent to incline 57. The notch 55 permits a user to recognize when the nail 12, targeting guide 14 and locking device 16 are completely interlocked together as the complementary locking element on the nail 12 is engaged with the locking device 16. Alternatively, one or more of the tabs 38 may include no cam surface at all, as seen with another exemplary embodiment to be described below. In this regard and as shown in FIG. 5C, a notched cam surface 53 may be included on the inner diameter of an alternative embodiment of an intramedullary nail 12a.

With particular reference to FIGS. 3A-3B and 4, coupling of the nail 12 to the locking device 16 is further facilitated by a lip 58 at the distal end 32 of locking device 16, which is configured to receive a shank portion 60 of the nail 12 therein. More particularly, lip 58 aids in centering or aligning the nail 12 relative to the locking device 16. In a similar fashion, a tapered recess 61 at the distal end 24 of the bore 26 in targeting guide 14 conform to and cooperate with the tapered shape of the head 30 of nail 12 to center and align the targeting guide 14 relative to the nail 1 2. The tapered recess 61 may include flats 62 that contact corresponding flats 63 on the head 30 of nail 12. As referred above, centering and alignment refer to the relative orientations of the axes 28 and 37 respectively of the barrel 18 and shaft 36 with a longitudinal axis 68 of the nail 12. While the exemplary targeting guide 16 is depicted as a hollow structure, those of ordinary skill in the art will appreciate that the targeting guide 1 6 may alternatively include a solid structure or any variation therebetween. In an alternative embodiment, the recess 62 may be omitted such that a flat annular surface surrounding the open end of the bore 61 contacts the nail 12.

Lip 58 represents a diametrically enlarged potion of a bore 61, which extends through at least a portion of the length of shaft 36. Bore 61 may be extended to cannulate the shaft 36 such that a guide wire (not shown) can be used in conjunction with the implantation system 10.

With renewed reference to FIGS. 1-2, coupling of the targeting guide 14 to the nail 12 is completed by lockingly engaging the targeting guide 14 to the locking device 16, which in turn lockingly engages the nail 12. To this end, the handle portion 40 of locking device 16 defines a stopping surface 70 to prevent disengagement or uncoupling of the targeting guide 14 from the nail 12. Moreover, rotation of the handle portion 40, along with shaft 36, about axis 37 thereof further secures the targeting guide 14 to the locking device 16. More particularly, rotation of the handle portion 40 in the direction of arrow 73 permits frictional locking engagement of a first ancillary locking element in the form of a protruding male locking projection 74 on the surface 70 thereof with a second ancillary locking element in the form of protruding cusped locking ridge 23 on the extending portion 20 of targeting guide 14. Engagement of male locking projection 74 and female cusped locking ridge 23 cooperate to further secure the angular orientation of the locking device 16 relative to the nail 12, thereby securing locking engagement of the nail 12, targeting guide 14, and locking device 16 to one another.

Those of ordinary skill in the art will appreciate that, alternatively, handle portion 40 may include a female cusped locking ridge while the extending portion 20 may include a cooperating male locking projection. Likewise, a different type of cooperating ancillary locking elements or no ancillary locking elements at all may be substituted for locking projection 74 and female cusped locking ridge 23.

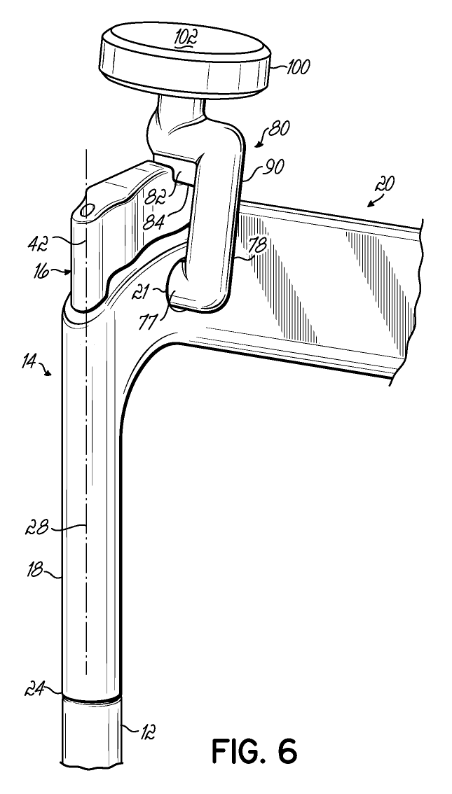

With reference to FIGS. 2 and 6, and as noted above, the extending portion 20 includes an opening 21, which further facilitates lockingly engagement of the implantation system 10. More particularly, opening 21 receives therethrough a leg portion 77 of a generally C-shaped base 78 of a redundant locking mechanism 80 that restricts axial and partial rotational movement of the targeting guide 14 relative to the locking device 16. Specifically, a lip 82 protrudes from the base 78 and engages a recess 84 on the handle portion 40 of locking device 16. Engagement of the lip 82 with recess 84, along with engagement of leg portion 77 within opening 21 jointly restrict axial movement (i.e., along axis 28 of the barrel 18) of the targeting guide 14 and locking device 1 6 relative to one another. Moreover, since locking device 16 restricts axial movement of the nail 12, the redundant locking mechanism 80 accordingly lockingly engages the targeting guide 14, locking device 16 and nail 12 relative to one another.

The redundant locking mechanism 80 further restricts, at least partially, rotational motion of the targeting guide 14 relative to the locking device 1 6. More particularly, center portion 90 of the C-shaped base 78 provides a stopping surface against which rotation of the handle portion 40, relative to targeting guide 14, in a direction toward center portion 90, is not physically possible or is substantially limited. Accordingly, the redundant locking mechanism 80 further secures the angular orientation of the locking device 16 relative to the targeting guide 14, thereby further securing locking engagement of the targeting guide 14, locking device 16 and nail 12 relative to one another.

With particular reference to FIG. 6, and in one aspect of the exemplary embodiment therein, the redundant locking mechanism 80 includes an impaction driver 100 in the form of a flat plate coupled to the C-shaped base 78. The impaction driver 100 accordingly provides an impaction surface 102 that can receive a driving force from an impacting tool such as hammer or mallet (not shown). The driving force is transferred, through the base 78, to the locking device 16 and targeting guide 14 engaging the nail 1 2, such that the force is, in turn, transferred to the nail 12 which can be driven into the desired intramedullary target in a patient's body. While the impaction driver 100 is depicted herein having a disc shape, other suitable shapes are similarly contemplated so long as they provide an impaction surface capable of receiving and transferring a driving force, as noted above.

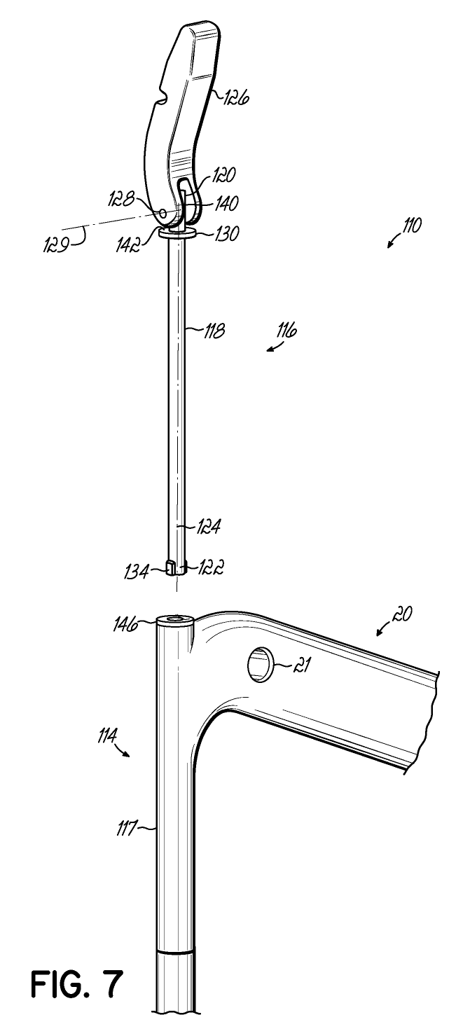

With reference to FIGS. 7-10 in which like reference numerals refer to similar features of FIGS. 1-6 and in accordance with another embodiment of the invention, an implantation system 110 is similar in most respects to implantation system 10 (FIGS. 1-6), the description of which may be referred to for an understanding of implantation system 110 as well. System 110 includes a targeting guide 114 and a locking device 116 defined by a shaft 118 having proximal and distal ends 120,122 and defining an axis 124. A handle portion 126 is pivotally coupled to the proximal end 120 and pivots about a pin 128 defining a pivot axis 129 that intersects the axis 124. A separating member such as one in the form of a washer e.g. a Belleville or cupped spring washer 130 is disposed and slidable about at least a portion of the shaft 118 adjacent the proximal end 120 thereof.

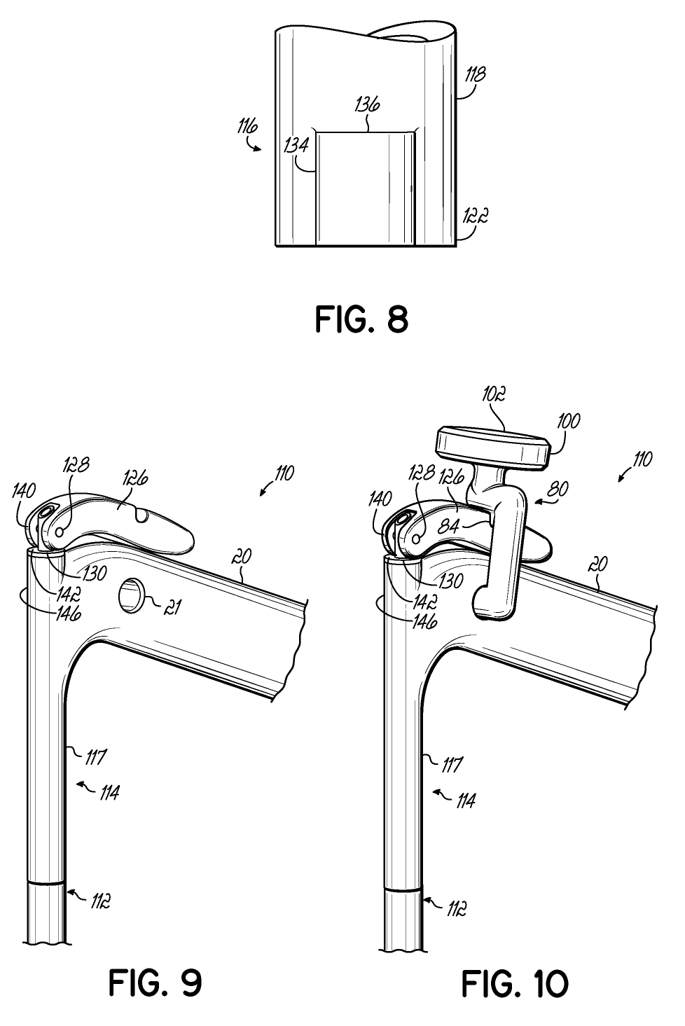

With particular reference to FIGS. 7-8, the distal end 122 is non-threaded and includes a pair of tabs 134 similar in most respects to tabs 38 of locking device 16 (FIG. 1). Unlike the exemplary embodiment of tabs 38, however, tabs 134 include no cam surfaces. Accordingly, each of the tabs 134 includes a top surface 136 that abuts against a corresponding confronting surface 52 of a locking portion 50 of nail 12 (FIG. 4) to restrict axial movement (along axis 124) of the locking device 11 6 relative the nail 1 2. This restriction occurs when the locking device 116 is rotated about axis 124 between first and second angular orientations relative to the nail 12 and tubular body 117 of the tubular guide 114 and respectively corresponding to the orientations depicted in FIGS. 7 and 9.

In order to restrict movement of the targeting guide 114 relative to the nail 12, targeting guide 114 includes at least one cam surface 140 on handle portion 126 that cooperates with a confronting surface 142 on cupped spring washer 130 to lockingly engage targeting guide 114 and nail 12 relative to one another. More particularly, rotation of the handle portion 126 about pin 128 from a vertical orientation (FIG. 7) to a horizontal orientation (FIG. 9) abuts cam surface 140 against confronting surface 142, thereby pushing cupped spring washer 130 against a proximal end 146 of the targeting guide 114. The pushing movement of the cupped spring washer 130 against proximal end 146 secures the axial position of the locking device 116 relative to the targeting guide 114, thereby further lockingly engaging the targeting guide 114, locking device 116 and nail 12 to one another.

In one aspect of this embodiment, abutting of the cam surface 140 against confronting surface 142 deflects the cupped spring washer toward the targeting guide 114, thereby generating a reaction force that biases the handle portion 126 away from the targeting guide 114. Moreover, this reaction force biases the nail 12 toward the targeting guide 114, thereby further lockingly engaging these two components.

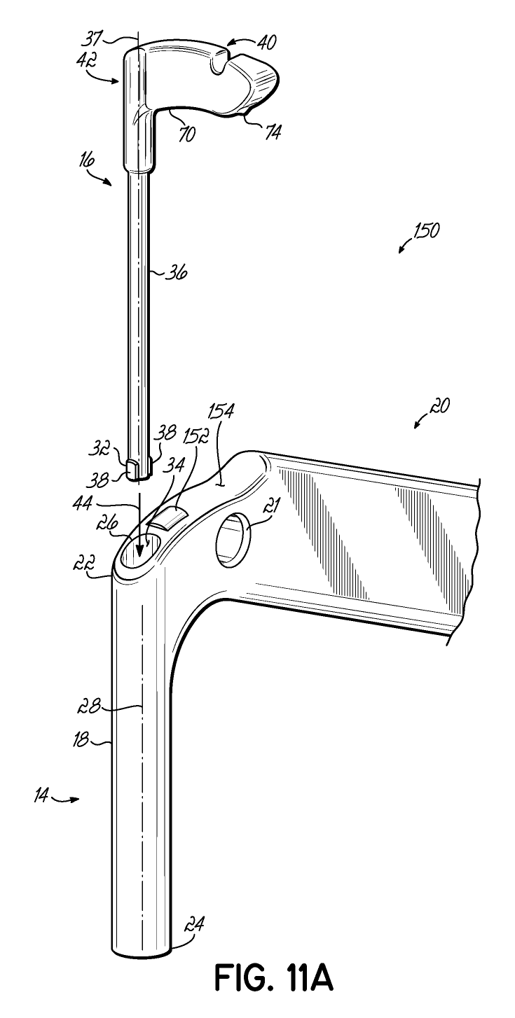

With particular reference to FIG. 10, implantation system 110 includes a redundant locking mechanism 80 to restrict axial and rotational movement of the locking device 116 relative to the targeting guide 114. More particularly, the redundant locking mechanism 80 engages an opening 21 on the extending portion 20 of targeting guide 114 and a recess 84 on handle portion 126 in fashions similar to that described for this aspect of the embodiment FIGS. 1-6, the description of which may be referred to for an understanding of this aspect as well. Similarly, the redundant locking mechanism 80 includes an impaction driver 100 having an impaction surface 102, similar in function to this aspect of the embodiment of FIGS. 1-6.

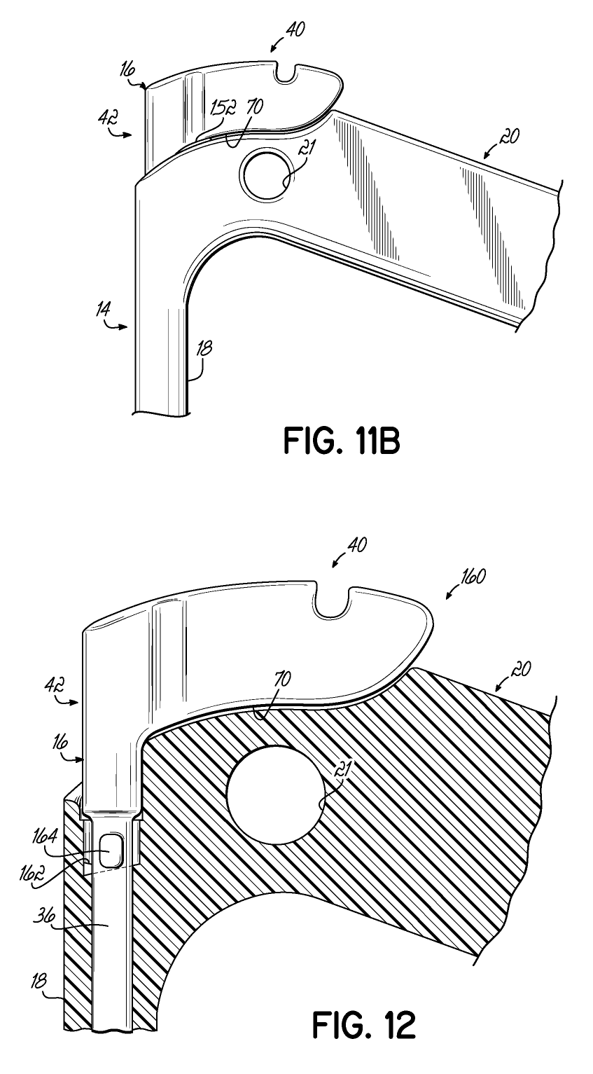

With reference to FIGS. 11A and 11B in which like reference numerals refer to similar features of FIGS. 1-6 and in accordance with another embodiment of the invention, an implantation system 150 is similar in most respects to implantation system 10 (FIGS. 1-6), the description of which may be referred to for an understanding of implantation system 150 as well. However, instead of cam surface 56 on tab 38, system 150 includes a cam surface 152 that is disposed on a surface 154 of the targeting guide 14. The locking device 16 includes tabs (not shown) identical in construction to tabs 134 (FIG. 8), which lack cam surfaces. When the handle portion 40 of the locking device 1 6 is rotated to engage the tabs with the corresponding confronting surface 52 of the locking portion 50 of nail 12 (FIG. 4) as shown in FIG. 11B, the stopping surface 70 of the locking device 16 rides on the cam surface 152 as the handle portion 40 is moved to couple the tabs with the nail 12, which pulls the nail 12 into engagement with the targeting guide 14 and locking device 16 and thereby creates an assembly. The cam surface 152 may be disposed anywhere on surface 154 of targeting guide 14that provides the requisite camming action required to lock the nail 1 2 with the targeting guide 14 and locking device 16.

A person having ordinary skill in the art will appreciate that a cam surface (not shown) similar to cam surface 152 may be disposed about the entrance to the bore 26 at end 22 of barrel 18. In one embodiment, the cam surface may be defined by the inclined plane of the barrel 18 about the entrance to the bore 26 at end 22 of barrel 18. In other words, the rotation of the handle portion 40 with the stopping surface 70 contacting this cam surface may cause the handle portion 40 to move along axis 37 in a direction that engages the tabs with the nail 12.

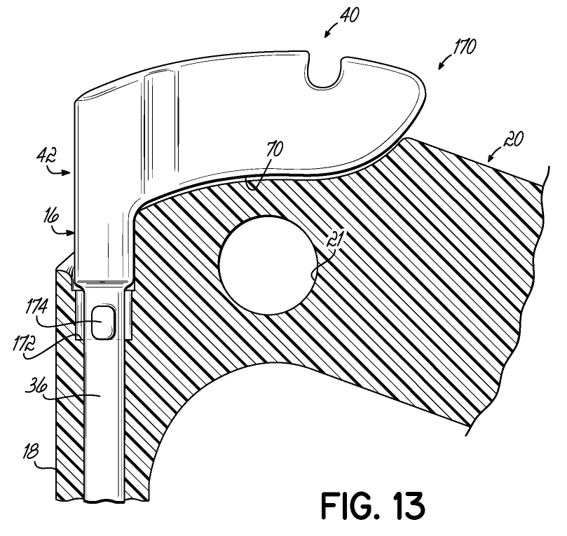

With reference to FIG. 12 in which like reference numerals refer to similar features of FIGS. 1-6 and in accordance with another embodiment of the invention, an implantation system 160 is similar in most respects to implantation system 10 (FIGS. 1-6), the description of which may be referred to for an understanding of implantation system 160 as well. However, instead of cam surface 56 on tab 38, system 160 includes a cam surface 162 in the form of a helical ramp that is disposed as a lip or ledge on the inner diameter of the bore 26 inside the barrel 18 and tabs 164. One of the tabs 164 is disposed on the diametrically opposite side of the shaft 36 and, as a result, is not visible in FIG. 12. The locking device 16 includes tabs (not shown) identical in construction to tabs 134 (FIG. 8), which lack cam surfaces.

The tabs 164 are located along axis 37 such that, when the locking device 16 is inserted fully into the targeting guide 14 and moved to secure the nail 12, the tabs 164 contact the cam surface 1 62. The cam surface 162 is oriented to face toward end 22 of the barrel 1 8. When the handle portion 40 is rotated to engage the tabs 134 with the corresponding confronting surfaces 52 of the locking portion 50 of nail 12 (FIG. 4), the tabs 164 ride on the cam surface 162. As tabs 164 slide across cam surface 162, the locking device 16 moves relative to the targeting guide 14 in a direction toward end 22 of barrel 18. Because the tabs 134 (FIG. 8) are engaged with the head 30 of nail 12, the nail 12 moves along axis 37 toward end 22 of barrel 18, which pulls the nail 12 into engagement with the targeting guide 14 and locking device 16 and, thereby, creates an assembly.

With reference to FIG. 13 in which like reference numerals refer to similar features of FIGS. 1-6 and in accordance with another embodiment of the invention, an implantation system 170 is similar in most respects to implantation system 10 (FIGS. 1-6), the description of which may be referred to for an understanding of implantation system 170 as well. However, instead of cam surface 56 on tab 38, system 170 includes a circular ledge or lip 172 that is disposed on the inner diameter of the bore 26 inside the barrel 18 and tabs 174 that are disposed at a location along axis 37 such that the tabs 174 contact the lip 172 when the locking device 16 is inserted fully into the targeting guide 14. The lip 172 is oriented to face toward end 22 of the barrel 18. One of the tabs 174 is disposed on the diametrically opposite side of the shaft 36 and, as a result, is not visible in FIG. 12. The tabs 174 are identical in construction to tabs 56 (FIG. 5).

The locking device 16 includes tabs (not shown) identical in construction to tabs 134 (FIG. 8), which lack cam surfaces. When the handle portion 40 is rotated to engage the tabs with the corresponding confronting surface 52 of the locking portion 50 of nail 12 (FIG. 4), the tabs 174 ride on the lip 172 as the handle portion 40 is moved to couple the tab with the nail 12, which pulls the nail 12 into engagement with the targeting guide 14 and locking device 16 and thereby creates an assembly.

Any one or more of the features depicted and described in regard to any of the above embodiments may be combined with any one or more of the features of another embodiment. Similarly, features described herein defining one feature on a component that cooperates with another feature on another component may be swapped in ways known to those of ordinary skill in the art. For example, and without limitation, a male twist-lock component may define a head of a nail while a cooperating female twist-lock component may define a distal end of a locking device. Likewise, a contemplated embodiment may include both of the types of cam surfaces described above, namely, the cam surface on each of the tabs of the locking device and the cam surface on a handle portion thereof.

While the invention has been illustrated by a description of various embodiments and while these embodiments have been described in considerable detail, it is not the intention of the applicants to restrict or in any way limit the scope of the appended claims to such detail. Additional advantages and modifications will readily appear to those skilled in the art. Thus, the invention in its broader aspects is therefore not limited to the specific details, representative apparatus and method, and illustrative example shown and described. Accordingly, departures may be made from such details without departing from the spirit or scope of applicants' general inventive concept.

What is claimed is:

1. An intramedullary implantation system comprising:
a nail including a head and a first locking element on said head;
a targeting guide including a tubular body with a first open end, a second open end, and a bore extending between said first and second open ends; and
a locking device including a shaft with a distal end and a non-threaded second locking element on said distal end, said distal end located proximate to said first open end of said bore of said targeting guide, when said shaft is positioned in said bore, so that said second locking element is engagable with said first locking element, and said locking device having a first angular orientation relative to said tubular body in which said first and second locking elements are engaged to secure said head of said nail to said targeting guide, and a second angular orientation relative to said tubular body in which said first locking element is disengaged from said second locking element.

2. The intramedullary implantation system of claim 1 wherein said second locking element includes at least one tab with a cam surface, said first locking element configured to contact said cam surface as said locking device is moved from the first angular orientation to the second angular orientation to move said first locking element in a direction toward said second open end of said bore.

3. The intramedullary implantation system of claim 1 wherein said second locking element includes at least one tab, said bore includes a cam surface, and said at least one tab configured to contact said cam surface as said locking device is moved from the first angular orientation to the second angular orientation to secure said first and second locking elements.

4. The intramedullary implantation system of claim 1 wherein said bore in said tubular body of said targeting guide includes a helical ledge defining a cam surface, and said shaft of said locking device includes at least one tab, said at least one tab configured to contact said cam surface as said locking device is moved from the first angular orientation to the second angular orientation to secure said first and second locking elements.

5. The intramedullary implantation system of claim 1 wherein said bore in said tubular body of said targeting guide includes a circular ledge, and said shaft of said locking device includes at least one tab with a cam surface, said cam surface on said at least one tab configured to contact said ledge as said locking device is moved from the first angular orientation to the second angular orientation to secure said first and second locking elements.

6. The intramedullary implantation system of claim 1 wherein said tubular body of said targeting guide includes a cam surface, said locking device configured to contact said cam surface as said locking device is moved from the first angular orientation to the second angular orientation to secure said first and second locking elements.

7. The intramedullary implantation system of claim 1 wherein said cam surface is located about said second open end.

8. The intramedullary implantation system of claim 1 further comprising:
a biasing member disposed between said targeting guide and said locking device, said biasing member configured to bias said nail toward said targeting guide.

9. The intramedullary implantation system of claim 1 wherein said locking device includes a first ancillary locking element and said targeting guide includes a second ancillary locking element, said second ancillary locking element configured to cooperate with said first ancillary locking element to bring said targeting guide and said locking device into locking engagement with one another.

10. The intramedullary implantation system of claim 9 wherein said first ancillary locking element is a protruding male member and said second ancillary locking element is a protruding female member.

11. The intramedullary implantation system of claim 9 wherein said locking device includes a handle portion coupled to said shaft and said targeting guide includes an extending portion coupled to said tubular body, said first ancillary locking element being disposed on said handle portion and said second ancillary locking element being disposed on said extending portion.

12. The intramedullary implantation system of claim 11 wherein said first ancillary locking element is configured to engage said second ancillary locking element when said locking device is rotated relative to said targeting guide.

13. The intramedullary implantation system of claim 1 wherein said bore includes a guiding surface configured to guide said second locking element toward said first locking element in a discrete orientation relative to said first locking element.

14. The intramedullary implantation system of claim 1 wherein said locking device includes a handle portion with a cam surface, said cam surface configured to handle portion configured about said pivot axis engages said cam surface with said confronting surface to bring said targeting guide and said locking device into locking engagement with one another.

15. The intramedullary implantation system of claim 1 wherein said first end of said bore includes an enlarged recess configured to at least partially receive said head of said nail.

16. An intramedullary nail comprising:
a longitudinal axis;
a shank disposed about said longitudinal axis; and
a head coupled to said shank, said head configured for inserting a locking element therein in a first angular orientation relative to said head and allow rotation of said locking element to a second angular orientation relative to said head to restrict said nail and said locking element from movement relative to one another along said longitudinal axis.

17. The intramedullary nail of claim 16 wherein said head includes a cam surface configured to engage the locking element.

18. An intramedullary implantation system comprising:
a nail including a head, said head including a first twist-lock element;
a targeting guide including a tubular body and a bore with first and second open ends; and
a locking device including a shaft configured to be positioned within said bore of said targeting guide, said shaft including a distal end with a second twist-lock element, said locking device having a first angular orientation relative to said tubular body in which said first and second twist-lock elements are engaged to secure said nail to said targeting guide, and a second angular orientation relative to said tubular body in which said first and second twist-lock elements are disengaged from each other.

19. The system of claim 18 wherein said locking device includes a first ancillary locking element and said targeting guide includes a second ancillary locking element configured to cooperate with said first ancillary locking element to bring said targeting guide and said locking device into locking engagement with one another.

20. The system of claim 18 wherein said locking device includes a handle portion pivotally coupled to said shaft, said handle portion including a pivot axis and a cam surface configured to apply a force against a confronting surface on said targeting guide, and rotation of said cam surface about said pivot axis brings said targeting guide and said locking device into locking engagement with one another.

21. A method of securing a targeting guide to an intramedullary nail, the method comprising:
coupling the targeting guide to a head of the intramedullary nail;
inserting a locking device through a bore of the targeting guide; and
rotating a non-threaded end of the locking device relative from a first angular orientation relative to the targeting guide to a second angular orientation relative to the targeting guide to secure the targeting guide to the intramedullary nail.

22. The method of claim 21 further comprising:
moving a contoured cam surface on the targeting guide in contact with a flat surface on the locking device as the locking device is rotated.

23. The method of claim 21 further comprising:
moving a flat surface on the targeting guide in contact with a contoured cam surface on the locking device as the locking device is rotated.

24. The method of claim 21 further comprising:
moving a flat surface on the locking device in contact with a contoured cam surface on the intramedullary nail as the locking device is rotated.

25. The method of claim 21 further comprising:
moving a contoured cam surface on the locking device in contact with a flat surface on the intramedullary nail as the locking device is rotated.
